(19) United States
(12) Patent Application Publication
(10) Pub. No.: US 2023/0033683 A1
(43) Pub. Date: Feb. 2, 2023

(54) INTERMITTENT CATHETERS

(71) Applicant: ConvaTec Limited, Deeside (GB)

(72) Inventors: Rachel Zimet Pytel, Las Vegas, NV (US); Neal Robert Carty, Las Vegas, NV (US); Lukas Kandrac, MIchalovc (SK)

(21) Appl. No.: 17/871,328

(22) Filed: Jul. 22, 2022

Related U.S. Application Data
(63) Continuation of application No. PCT/GB2022/051921, filed on Jul. 22, 2022.
(60) Provisional application No. 63/203,587, filed on Jul. 27, 2021.

(51) Int. Cl. A61M 25/00; A61L 29/14; A61L 29/08

(52) U.S. Cl. CPC A61M 25/0017; A61M 25/0045; A61M 25/002; A61L 29/141; A61L 29/085; A61M 2025/0047

(57) ABSTRACT

The invention provides an intermittent catheter comprising a hollow polymeric tubular body comprising a base polymer and further comprising an amphiphilic additive at and/or on an outer surface of the body, wherein one or both of the base polymer and the additive are independently cross-linked and/or the base polymer and additive are cross-linked with each other.

### CROSS REFERENCE TO RELATED DISCLOSURES

The present disclosure is a continuation of International Application No. PCT/GB2022/051921 filed on Jul. 22, 2022 and claims the benefit of U.S. Provisional Application No. 63/203,587 filed on Jul. 27, 2021, the contents of which are incorporated herein by reference.

### TECHNICAL FIELD OF THE INVENTION

The present invention relates to cross-linked intermittent catheters comprising a base polymer and an additive, to methods of manufacturing said intermittent catheters, and to the use of cross-linking to reduce migration of additives from intermittent catheters.

### BACKGROUND TO THE INVENTION

Intermittent urinary catheterisation is a process involving insertion of a urinary catheter through an individual's urethra and into their bladder, where it is retained to empty the bladder of urine for only the time period that is required for emptying, after which the catheter is removed. The process differs from long-term catheterisation, which makes use of an indwelling or Foley catheter that is inserted into the bladder for long periods of time (several days to months) to discharge the residual urine of the bladder continuously throughout the day.

Intermittent catheterisation is often used by patients suffering from abnormalities of the urinary system, resulting in urinary incontinence and/or a lack of control in permitting voluntary urination. Such individuals would typically make use of intermittent catheters several times a day.

Intermittent catheters are useful devices, providing users with independence and freedom to self-catheterise as and when required, without having to rely on trained personnel to be present. This, however, increases the need for intermittent catheters to be user friendly: in particular, both easy to insert and remove with minimum discomfort caused, and safe to use with features for minimising risk of infection. Users often report experiencing pain and discomfort upon insertion and/or removal of intermittent catheters. Users have, for instance, reported experiencing bladder spasms, burning sensations, and bleeding. Urinary tract infections (UTI) are also common in individuals who practice intermittent catheterisation.

Surface coatings and additives for intermittent catheters have been used to help in alleviating these issues. However, intermittent catheter surface coatings and additives have the tendency to migrate out of the catheter with time and use, which causes the surface of the catheter to become less lubricious. Often coatings and additives may migrate out of a catheter even when the catheter is not in use, such as when it may be packaged in a storage or transportation medium.

In use, scraping of the catheter surface may occur, further accelerating the removal of any surface coatings or additives.

Furthermore, when a person uses an intermittent catheter, some of the coating may be left inside the user's body, which can be harmful and thus unacceptable.

To address some of the above problems, US patents U.S. Pat. No. 10,058,638 B2 and U.S. Pat. No. 9,186,438 B2 describe the use of a catheter containing a polymer mixture of a thermoplastic or thermocuring polymer base material and an amphiphilic block copolymer lubricious additive. The amphiphilic block copolymer contains both a hydrophobic and hydrophilic portion. The hydrophilic portion diffuses to the surface of the catheter due to incompatibility with the hydrophobic base material and provides for a lubricious surface coating. Interactions between the hydrophobic portion of the amphiphilic molecule and the base material assist in reducing migration of the amphiphilic molecule from the catheter. Whilst this method has its advantages, there exists a need for catheters with further safeguards to reduce migration of surface coatings and additives from the catheter.

There is a particular need for intermittent catheters that can be packaged in a storage or transportation medium with reduced migration of surface coatings and additives from the catheters.

It is an aim of embodiments of the present invention to address one or more of the above problems by providing an intermittent catheter, suitable for self-catheterisation use, which provides one or more of the following advantages:
A lubricous, non-stick surface making the intermittent catheter easier to insert and remove.
Retention of a lubricious, non-stick surface even when the surface is scraped.
Reduced migration of coatings and additives in the intermittent catheter.
Reduced migration of coatings and additives out of the intermittent catheter, which have the potential to enter a person's body upon use of the catheter.
Reduced migration of coatings and additives out of the intermittent catheter when the catheter is not in use, such as when it is packaged in a storage or transportation medium.

It is also an aim of embodiments of the invention to overcome or mitigate at least one problem of the prior art, whether expressly described herein or not.

### SUMMARY OF THE INVENTION

According to one aspect of the invention, there is provided an intermittent catheter comprising a hollow polymeric tubular body comprising a base polymer and further comprising an amphiphilic additive at and/or on an outer surface of the body, wherein one or both of the base polymer and the additive are independently cross-linked and/or the base polymer and additive are cross-linked with each other.

Such an additive at and/or on an outer surface of the body provides high lubricity to the surface, making the intermittent catheter easier and less painful to use, especially for individuals practicing self-catheterisation. Additionally, the cross-links increase the difficulty for the additive to migrate, particularly out of the intermittent catheter. This allows for the intermittent catheter to retain its lubricious surface for longer and even when the surface of the catheter is scraped. Advantageously, the intermittent catheter is also able to retain its lubricious surface after longer storage periods in a storage or transportation medium, without loss of lubricious additive into the surrounding solution.

The additive may be incorporated into the base polymer and/or be located as a layer or coating on the base polymer.

In some embodiments, the base polymer is cross-linked. In some embodiments, the base polymer is both independently cross-linked and cross-linked with the additive, and the additive is not independently cross-linked. In other embodiments, the base polymer is independently cross-linked but not cross-linked with the additive, and the additive is not independently cross-linked.

In some embodiments, the additive is cross-linked. In some embodiments, the additive is both independently cross-linked and cross-linked with the base polymer, and the base polymer is not independently cross-linked. In other embodiments, the additive is independently cross-linked but not cross-linked with the base polymer, and the base polymer is not independently cross-linked.

In some embodiments, the base polymer and additive are both independently cross-linked but not cross-linked with each other. In other embodiments, the base polymer and additive are not independently cross-linked but are cross-linked with each other. In other embodiments, both the base polymer and additive are independently cross-linked and cross-linked with each other.

In some embodiments, the base polymer is cross-linked at least at and/or on the outer surface. This is advantageous as it further restricts migration of the additive out from the surface.

In some embodiments, the base polymer comprises a polymer selected from the group comprising: polyvinyl chloride, polytetrafluoroethylene, polyolefins, latex, silicones, synthetic rubbers, polyurethanes, polyesters, polyacrylates, polyamides, thermoplastic elastomeric materials, styrene block copolymers, polyether block amide, thermoplastic vulcanizates, thermoplastic copolyesters, thermoplastic polyamides, styrene-butadiene copolymer (SBC), styrene-ethylene-butylene-styrene copolymer (SEBS), and water disintegrable or enzymatically hydrolysable material, or combinations, blends or copolymers of any of the above materials.

In preferred embodiments, the base polymer comprises a polymer selected from the group comprising: polyolefins, polyesters, polyacrylates, polyamides, thermoplastic elastomeric material, polyether block amide, thermoplastic vulcanizates, thermoplastic copolyesters, thermoplastic polyamides, fluororubber, and water disintegrable or enzymatically hydrolysable material or combinations, blends or copolymers of any of the above materials.

In some embodiments, said water disintegrable or enzymatically hydrolysable material comprises a material of the group comprising: polyvinyl alcohol, extrudable polyvinyl alcohol, polyacrylic acids, polylactic acid, polyesters, polyglycolide, polyglycolic acid, poly lactic-co-glycolic acid, polylactide, amines, polyacrylamides, poly(N-(2-Hydroxypropyl) methacrylamide), starch, modified starches or derivatives, amylopectin, pectin, xanthan, scleroglucan, dextrin, chitosans, chitins, agar, alginate, carrageenans, laminarin, saccharides, polysaccharides, sucrose, polyethylene oxide, polypropylene oxide, acrylics, polyacrylic acid blends, poly(methacrylic acid), polystyrene sulfonate, polyethylene sulfonate, lignin sulfonate, polymethacrylamides, copolymers of aminoalkyl-acrylamides and methacrylamides, melamine-formaldehyde copolymers, vinyl alcohol copolymers, cellulose ethers, poly-ethers, polyethylene oxide, blends of polyethylene-polypropylene glycol, carboxymethyl cellulose, guar gum, locust bean gum, hydroxypropyl cellulose, vinylpyrrolidone polymers and copolymers, polyvinyl pyrrolidone-ethylene-vinyl acetate, polyvinyl pyrrolidone-carboxymethyl cellulose, carboxymethyl cellulose shellac, copolymers of vinylpyrrolidone with vinyl acetate, hydroxyethyl cellulose, gelatin, poly-caprolactone, poly(p-dioxanone), or combinations, blends or co-polymers of any of the above materials.

In other preferred embodiments, the base polymer comprises a polymer selected from the group comprising: polyolefins, polyvinyl chloride, polyurethane, styrene-butadiene copolymer (SBC), styrene-ethylene-butylene-styrene copolymer (SEBS), and thermoplastic elastomeric material or combinations, blends or copolymers of any of the above materials.

In some preferred embodiments, the base polymer comprises a polyolefin, such as polyethylene and/or polypropylene.

In some preferred embodiments, the base polymer comprises a thermoplastic elastomeric material. The base polymer may comprise a thermoplastic polyolefin.

In some embodiments, the base polymer comprises silane groups and the base polymer is cross-linked through Si—O—Si bonds between the silane groups and water. The silane groups may comprise trialkoxysilane groups or trialkylsilane groups.

At least some of the additive is at or on the outer surface of the body. By “at the outer surface”, it is meant that at least a portion of the additive forms part of the surface or protrudes from the surface. In some embodiments part of the additive is retained or anchored in the body while part of the additive forms part of or protrudes from the outer surface of the body.

The outer surface may comprise at least one surface of the group comprising: the external facing surface of the body, the lumen of the body, and any eyelets present on the body. In preferred embodiments, the outer surface is the external-facing surface of the body and/or the inner lumen. In some embodiments, the outer surface may comprise the external-facing surface of the body of the catheter, the inner lumen, and the eyelets.

Amphiphilic additives can provide an effective lubricious catheter surface. The additive may be polymeric or oligomeric. Amphiphilic additives are particularly useful as lubricious additives and in embodiments in which the base polymer is hydrophobic or generally hydrophobic, such as a polyolefin, the amphiphilic additives will diffuse towards and to the outer surface due to incompatibility of the hydrophilic portion of the amphiphilic additive with the hydrophobic base polymer. At the same time, cross-linking the base polymer, additive or the additive to the base polymer, ensures that complete migration of the additive out of the base polymer is reduced or prevented. When the hydrophilic portion of an amphiphilic additive is present at or on the outer surface it enables wetting of the outer surface simply by applying water or wiping with a wet wipe, to create a lubricious coating, while the cross-linking ensures that the additive remains connected to or presented on or from the outer surface for the maximum time and mitigates removal by friction or abrasion.

In some embodiments, the hydrophilic portion of at least some of the additive may protrude from or form part of the outer surface of the body, while at least part of the hydrophobic portion may be retained or anchored within the polymer body.

The additive may be concentrated at or on the outer surface of the body. For example, at least 25%, 30%, 35%, 40%, 45%, 50%, 55%, 60%, 65%, 70%, 75% or at least 80% of the number of molecules of the additive may be at or on the outer surface of the body.

In some embodiments, at least 25%, 30%, 35%, 40%, 45%, 50%, 55%, 60%, 65%, 70%, 75% or at least 80% of the number of molecules of additive may have hydrophilic portions that are at or on the outer surface of the body.

Therefore, in some embodiments, there will be a portion of amphiphilic additive within the body of the intermittent catheter, for which no part is at or on the outer surface of the body, and another portion of the amphiphilic additive at or on the outer surface of the body. In such embodiments, cross-linking of the additive and/or base polymer provides an insoluble matrix of additive and/or polymer of the body at or on the surface, which prevents or reduces migration of not only any cross-linked additive, but also any non-cross-linked additive which may be wholly within the body of the catheter, out of the catheter. Such embodiments may be useful, for example, if there is scraping of the catheter during use, which may remove at least some of the amphiphilic additive from the surface of the catheter, by enabling further additive to then migrate towards the surface where additive has been removed, regenerating the surface for continued use.

In some embodiments, the additive is an A-B block copolymer comprising a hydrophobic hydrocarbon A-block and a hydrophilic B-block. The hydrophobic portion (A-block) may comprise a carbon chain of at least 5 carbon atoms, or at least 10, 15, 20, 25, 30, 35, 40 carbon atoms. The hydrophobic portion may preferably comprise a carbon chain of between 12-52 carbon atoms, or of between 20-52 carbon atoms.

In some embodiments, the A-block comprises a hydrocarbon chain block of the formula CH₃CH₂(CH₂CH₂)ₐ. The value of “a” may be between 5-25; for instance, “a” may be 5, 6, 7, 8, 9, 10, 11, 12, 13, 14, 15, 16, 17, 18, 19, 20, 21, 22, 23, 24, or 25, or a half integer of any of the above values. The value of “a” may preferably be between 9-25; for instance, “a” may be 9, 10, 11, 12, 13, 14, 15, 16, 17, 18, 19, 20, 21, 22, 23, 24, or 25, or a half integer of any of the above values.

In some embodiments, the B-block is a hydrophilic oligomer, i.e. a homo- or co-oligomer, comprising between 2 and 10 monomer units. The monomer units may be selected from the group comprising: alkylene oxides, alkylene glycols, epihalohydrins, unsaturated carboxylic acids, alkylene imines, lactones, vinyl alcohol, and vinyl alkanoates. The monomer units may be preferably selected from the group comprising: ethylene oxide, propylene oxide, ethylene glycol, propylene glycol, epichlorohydrin, acrylic acid, methacrylic acid, ethylene imine, caprolactone, vinyl alcohol, and vinyl acetate. In some embodiments the monomer units comprise alkylene oxide groups independently selected from ethylene oxide and propylene oxide, and in preferred embodiments all of the monomer units are ethylene oxide or all of the monomer units are propylene oxide.

In some embodiments, the additive (preferably the A-B block copolymer defined hereinabove) comprises poly(alkylene oxide) groups, preferably polyethylene oxide, and the additive is cross-linked through non-covalent bonds between the poly(alkylene oxide) groups and a complexing agent.

The complexing agent may be selected from one or more of the group comprising: a urea, a cyclodextrin, and a poly(unsaturated carboxylic acid), or combinations and/or derivatives thereof. The urea is preferably urea per se. The cyclodextrin may be selected from the group comprising: α-cyclodextrin, β-cyclodextrin, and γ-cyclodextrin; preferably α-cyclodextrin. The poly(unsaturated carboxylic acid) may preferably comprise poly(methacrylic acid) or a copolymer thereof. The copolymer may comprise a copolymer of poly(methacrylic acid) and an acrylate polymer, and preferably poly(methacrylic acid-co-methyl methacrylate).

In some embodiments, the additive is located at and/or on at least 50, 55, 60, 65, 70, 75, 80, 85, 90, 95, 96, 97, 98 or at least 99% of the outer surface area of the polymeric tubular body, preferably at least 75% or at least 90% of the outer surface area of the polymeric tubular body or between 75% and 100% of the outer surface area.

In some embodiments, the additive comprises a concentration of at least 0.1, 0.2, 0.3. 0.4. 0.5, 0.75, 1, 2, 3, 4, 5, 10, 15 or at least 20%, preferably between 0.1-20%, and more preferably between 0.5-15% or 0.5-5% by weight of the combination of base polymer and additive.

In some embodiments, the intermittent catheter comprises a layer of the additive on and/or comprising the outer surface of the body. The layer may comprise a thickness of at least 5, 10, 15, 20, 25, 30, 35, 40, 45 or at least 50 μm. The layer may comprise a thickness of no more than 1 mm or no more than 750, 700, 650, 600, 550, 500, 450, 400 or 350 μm. A preferred thickness is between 50 and 300 μm. The layer may comprise an additive concentration of greater than 5% by weight of the combination of base polymer and additive.

In some embodiments, the outer surface of the polymeric tubular body comprises a separate or further lubricating agent or bacteria-repellent agent at and/or on the surface, in addition to the additive. The separate or further lubricating agent or bacteria-repellent agent may be cross-linked or otherwise bonded at and/or on the surface.

In some embodiments, said further lubricating agent or bacteria-repellent agent is formed from a coating material selected from the group comprising: silver-based, polytetrafluoroethylene, hydrogel, silicone, lecithin, salicylic acid, minocycline, rifampin, fluorinated ethylene propylene, polyvinylidone, polyvinyl compounds, polylactames, polyvinyl pyrrolidones, polysaccharides, heparin, dextran, xanthan gum, derivatised polysaccharides, hydroxy propyl cellulose, methyl cellulose, polyurethanes, polyacrylates, polyhydroxyacrylates, polymethacrylates, polyacrylamides, polyalkylene oxides, polyethylene oxides, polyvinyl alcohols, polyamides, polyacrylic acid, hydroxy ethylmethyl acrylate, polymethylvinyl ether, maleinic acid anyhydride, penicillin, neomycin sulfate, cephalothin, Bacitracin, phenoxymethyl penicillin, lincoymycin hydrochloride, sulfadiazine, methyl sulfadiazine, succinoylsulfathiazole, phthalylsulfathiazde, sulfacetamine, procaine penicillin, streptomycin, aureomycin, terramycin, terramycin, quaternary ammonium halides, cetyl pyridinium chloride, triethyl dodecyl ammonium bromide, hexachlorophene and nitrofurazone, or any combination thereof.

According to a second aspect of the invention, there is provided a method of manufacturing an intermittent catheter, the method comprising the steps of:
a) Providing a base polymer and an amphiphilic additive; and
b) Forming a hollow polymeric tubular catheter body comprising the base polymer and amphiphilic additive

wherein the method further comprises the step of independently cross-linking one or both of the base polymer and the amphiphilic additive and/or cross-linking the base polymer and the amphiphilic additive with each other.

According to a third aspect of the invention, there is provided a method of reducing migration of an amphiphilic additive from an intermittent catheter, the intermittent catheter comprising a polymeric tubular body comprising a base polymer and an amphiphilic additive, the method comprising the step of independently cross-linking one or both of the base polymer and the amphiphilic additive and/or cross-linking the base polymer and the amphiphilic additive with each other.

In preferred embodiments, the method comprises reducing migration of the additive out of the intermittent catheter.

The intermittent catheter referred to in the second and third aspects of the invention may comprise any intermittent catheter of the first aspect of the invention. Statements of invention relating to the intermittent catheter of the first aspect of the invention may also be applied to the second and third aspects of the invention.

The following statements apply to the second and third aspects of the invention.

The base polymer and/or additive, preferably both, may be provided in granulate or powder form. The granulate or powder base polymer and additive may be melt-extruded or injection-moulded to form the hollow polymeric tubular intermittent catheter body.

In some embodiments, the method comprises cross-linking the base polymer. In some embodiments, the method comprises both independently cross-linking the base polymer and cross-linking the base polymer with the additive. In other embodiments, method comprises independently cross-linking the base polymer but not cross-linking the base polymer with the additive.

In some embodiments, the method comprises independently cross-linking the additive. In some embodiments, the method comprises both independently cross-linking the additive and cross-linking the additive with the base polymer. In other embodiments, the method comprises independently cross-linking the additive but not cross-linking the additive with the base polymer.

In some embodiments, the method comprises independently cross-linking both the base polymer and the additive but not cross-linking the base polymer and additive with each other. In other embodiments, the method comprises cross-linking the base polymer and additive with each other but not independently cross-linking the base polymer or additive. In other embodiments, the method comprises independently cross-linking both the base polymer and additive and cross-linking the base polymer and the additive with each other.

In some embodiments, the method comprises cross-linking one or both of the base polymer and the additive independently before cross-linking the base polymer and the additive with each other.

In other embodiments, the method comprises cross-linking one or both of the base polymer and the additive independently after cross-linking the base polymer and the additive with each other.

In some embodiments, the method comprises cross-linking one or both of the base polymer and the additive independently at the same time as cross-linking the base polymer and the additive with each other.

In some embodiments the method comprises cross-linking only the base polymer or only the additive.

In some embodiments, the method comprises cross-linking one or both of the base polymer and the additive independently and/or cross-linking the base polymer and the additive with each other during co-granulation of the base polymer with the additive.

In some embodiments, the method comprises the step of cross-linking one or both of the base polymer and the additive independently and/or cross-linking the base polymer and the additive with each other during and/or after extrusion or injection moulding of a mixture comprising the base polymer and the additive. Cross-linking may be performed during, after, or both during and after extrusion or injection moulding. The method may comprise cross-linking one or both of the base polymer and the additive independently during extrusion or injection moulding and cross-linking the base polymer and the additive with each other after extrusion or injection moulding. Alternatively, the method may comprise cross-linking the base polymer and the additive with each other during extrusion or injection moulding and cross-linking one or both of the base polymer and the additive independently after extrusion or injection moulding.

In some embodiments, the method comprises the step of cross-linking one or both of the base polymer and the additive independently and/or cross-linking the base polymer and the additive with each other during and/or after co-extrusion of a layer of the additive on an outer surface of the base polymer.

In some embodiments, the method comprises the step of adding at least one cross-linker to one or both of the base polymer and the additive.

The at least one cross-linker may comprise one or both of an unsaturated monomer and a free radical.

The free radical may be produced by controlled/living radical polymerisation techniques. These techniques are known to a skilled person in the art and employ the principle of an equilibrium between free radicals and various types of dormant species (depending on the specific type of polymerisation technique employed.

The controlled/living radical polymerisation techniques include nitroxide-mediated polymerisation, reversible addition fragmentation transfer polymerisation (RAFT) and atom transfer radical polymerisation (ATRP). Anionic polymerisation may also be possible, although polymerisation is more sensitive to moisture and oxygen as impurities so may be less favourable than other controlled/living radical polymerisation processes.

More detailed descriptions of polymerisation mechanisms and related chemistry is discussed for nitroxide-mediated polymerisation (Chapter 10, pages 463 to 522). ATRP (Chapter 11, pages 523 to 628) and RAFT (Chapter 12. pages 629 to 690) in the Handbook of Radical Polymerization, edited by Krzysztof Matyjaszewski and Thomas P. Davis, 2002, published by John Wiley and Sons Inc.

When the polymer is prepared from anionic polymerisation techniques, initiators include. for example, hydrocarbyllithium initiators such as alkyilithium compounds (e.g., methyl lithium n-butyl lithium, sec-butyl lithium), cycloalkyllithium compounds (e.g., cyclohexyl lithium and aryl lithium compounds (e.g., phenyl lithium, 1-methylstyryl lithium, n-tolyl lithium, naphyl lithium 1,1-diphenyl-3-methylpentyl lithium. Also, useful initiators include naphthalene sodium, 1,4-disodio-1,1,4,4-tetraphenyibutane, diphenylmethyl potassium or diphenylmethyisodium.

The polymerisation process may also be carried out in the absence of moisture and oxygen and in the presence of at least one inert solvent. In one embodiment anionic polymerisation is conducted in the absence of any impurity which is detrimental to an anionic catalyst system. The inert solvent includes a hydrocarbon, an aromatic solvent or ether. Suitable solvents include isobutane, pentane, cyclohexane, benzene, toluene, xylene, tetrahydrofuran, diglyme, tetraglyme, orthoterphenyl, biphenyl, decalin or tetralin.

The anionic polymerisation process may be carried out at a temperature of 0° C. to −78 ° C.

A more detailed description of process to prepare polymers from an anionic process is discussed in Textbook of Polymer Science, edited by Fred W. Billmeyer Jr., Third Edition, 1984, Chapter 4, pages 88-90.

The controlled/living polymerisation processes leave a residue of reagente polymer chain such as (nitroxyl group from nitroxide-mediated), or a halogen from ATRP, thiocarbonylthio group from RAFT.

lit one embodiment it is desirable to remove the residue i.e., remove the nitroxyl halogen or thiocarbonylthio group. Processes are known to a skilled person to remove such groups, and the disclosure in EP2791184 provides a solution to remove thiocarbonylthio groups. Other such techniques are described, for example, in Chong et at, Macromolecules 2007, 40, 4446-4455; Chong et al, Aust. J. Chem. 2006, 59, 755-762; Postma et al, Macromolecules 2005, 38, 5371-5374; Moad et al, Polymer International 60, no. 1, 2011, 9-25; and Wilcock et al, Polym. Chem 2010, 1, 149-157.

In one embodiment The at least one cross-linker may comprise one or both of an unsaturated monomer and a nitroxyl free radical (common when nitroxide-mediated polymerisation is employed). The unsaturated monomer may comprise a monofunctional monomer and/or multifunctional monomer, such as a multifunctional olefin. The unsaturated monomer may be selected from the group comprising: acetylene, triallylisocyanurate (TAIC), trimethylol-propane-trimethacrylate (TMPTMA), triallylcyanurate (TAC), trimethylol-propane-triacrylate (TMPTA), hexakisalylaminocyclotriphosphazatrine (HAAP), maleic anhydride (MA), and poly(ethylene glycol) diacrylate (PEGDA), or combinations and/or derivatives thereof The nitroxyl free radical may comprise 2,2,6,6-tetramethylpiperidin-1-yl)oxyl free radical (TEMPO) and/or a derivative thereof, which may optionally be selected from the group comprising: 4-hydroxy-2,2,6,6-tetramethylpiperidine-1-oxyl free radical (HO-TEMPO) and 4-benzoyloxy-2,2,6,6-tetramethylpiperidine-1-oxyl free radical (BzO-TEMPO), or combinations and/or derivatives thereof.

In ATRP polymerisation, groups that may be transferred by a radical mechanism include halogens (from a halogen-containing compound) or various ligands. A more detailed review of groups that may be transferred is described in U.S. Pat. No. 6,391,996.

Examples of a halogen-containing compound that may be used in ATRP polymerisation include benzyl halides such as p-chloromethylstyrene, α-dichloroxylene, α,α-dichloroxylene, α,α-dibromoxylene, hexakis(α-bromomethyl)benzene, hexakis(α-bromomethyl)benzene, benzyl chloride, benzyl bromide, 1-bromo-1-phenylethane and 1-chloro-1-phenylethane; carboxylic acid derivatives which are halogenated at the α-position, such as propyl 2-bromopropionate, methyl 2-chloropropionate, ethyl 2-chloropropionate, methyl 2-bromopropionate, and ethyl 2-bromoisobutyrate tosylhalides such as p-toluenesulfonyl chloride; alkylhalides such as tetrachloromethane, tribromomethane, 1-vinylethyl chloride, and 1-vinylethyl bromide; and halogen derivatives of phosphoric acid esters, such as dimethylphosphoric acid.

In one embodiment when the halogen compound is employed, a transition metal such as copper is also present. The transition metal may be in the form of a salt. The transition metal is capable of forming a metal-to-ligand bond and the ratio of ligand to metal depends on the dentate number of the ligand and the co-ordination number of the metal. The ligand may be a nitrogen or phosphorus-containing ligand.

Examples of a suitable ligand include triphenylphosphine, 2,2-bipyridine, alkyl-2,2-bipyridine, such as 4,4-di-(5-heptyl)-2,2-bipyridine, tris(2-aminoethyl)amine (TREN), N,N,N′,N′,N″-pentamethyldiethylenetriamine, 4,4-do-(5-nonyl)-2,2-bipyridine, 1,1,4,7,10,10-hexamethyltriethylenetetramine and/or tetramethylethylenediamine. Further suitable ligands are described in, for example, International Patent application WO 97/47661. The ligands may be used individually or as a mixture. In one embodiment the nitrogen containing ligand is employed in the presence of copper. In one embodiment the ligand is phosphorus-containing with triphenyl phosphine (PPh₃) a common ligand. A suitable transition metal for a triphenyl phosphine ligand includes Rh, Ru, Fe, Re, Ni or Pd.

In RAFT polymerisation, chain transfer agents are important. A more detailed review of suitable chain transfer agents RAFT polymerisation, as described in International Patent Publication Nos. WO 98/01478, WO 99/31144 and WO 10/83569, is a polymerisation technique that exhibits characteristics associated with living polymerisation.

Examples of a suitable RAFT chain transfer agent include benzyl 1-(2-pyrrolidinone)carbodithioate, benzyl(1,2-benzenedicarboximido) carbodithioate, 2-cyanoprop-2-yl 1-pyrrolecarbodithioate, 2-cyanobut-2-yl 1-pyrrolecarbodithioate, benzyl 1-imidazolecarbodithioate, N,N-dimethyl-S-(2-cyanoprop-2-yl)dithiocarbamate, N,N-diethyl-S-benzyl dithiocarbamate, cyanomethyl 1-(2-pyrrolidone) carbodithoate, cumyl dithiobenzoate, 2-dodecylsulphanyithiocarbonylsulphanyl-2-methyl-propionic acid butyl ester, O-phenyl-S-benzyl xanthate, N,N-diethyl S-(2-ethoxy-carbonylprop-2-yl)dithiocarbamate, dithiobenzoic acid, 4-chlorodithiobenzoic acid, O-ethyl-S-(1-phenylethyl)xanthtate, O-ethyl-S-(2-(ethoxycarbonyl)prop-2-yl)xanthate, O-ethyl-S-(2-cyanoprop-2-yl)xanthate, O-ethyl-S-(2-cyanoprop-2-yl)xanthate, O-ethyl-S-cyanomethyl xanthate, O-pentafluorophenyl-S-benzyl xanthate, 3-benzylthio-5,5-dimethylcyclohexene-2-ene-1-thione or benzyl 3,3-di(benzylthio)prop-2-enedithioate, S,S′-bis-(α,α′-disubstituted-α″-acetic acid)-trithiocarbonate, S,S′-bis-(α,α′-disubstituted-α″-acetic acid)-trithiocarbonate or S-allyl-S′-(α,α′-disubstituted-α″-acetic acid)-trithiocarbonates, benzyl dithiobenzoate, 1-phenylethyl dithiobenzoate, 2-phenylprop-2-yl dithiobenzoate, 1-acetoxyethyl dithiobenzoate, hexakis(thiobenzoylthioethyl)benzene, 4-bis(thiobenzoylthiomethyl)benzene, 1,2,4,5-tetrakis(thiobenzoylthiomethyl)benzene, 1,4-bis-(2-(thiobenzoyithio)-prop-2-yl)benzene, 1-(4-methoxyphenypethyl dithiobenzoate, benzyl dithioacetate, ethoxycarbonyl ethyl dithioacetate, 2-(ethoxycarbonyl)prop-2-yl dithiobenzoate, 2,4,4-trimethylpent-2-yl dithiobenzoate, 2-(4-chlorophenyl)prop-2-yl dithiobenzoate, 3-vinyibenzyl dithiobenzoate, 4-vinylbenzyl dithiobenzoate, S-benzyl diethoxyphosphinyldithioformate, tert-butyl trithioperbenzoate, 2-phenylprop-2-yl 4-chlorodithtobenzoate, 2-phenylprop-2-yl 1-dithionaphthalate, 4-cyanopentanoic acid dithiobenzoate, dibenzyl tetrathioterephthalate, dibenzyl trithiocarbonate, carboxymethyl dithiobenzoate or polyethylene oxide) with dithiobenzoate end group or mixtures thereof.

In one embodiment a suitable RAFT chain transfer agent includes 2-Dodecylsulfanylthiocarbonylsulfanyl-2-methyl-propionic acid butyl ester, cumyl dithiobenzoate or mixtures thereof.

A discussion of the polymer mechanism of RAFT polymerisation is shown on page 664 to 665 in section 12.4.4 of Matyjaszewski et al.

The method may comprise adding the at least one cross-linker to one or both of the base polymer and the additive before or during extrusion or injection moulding of the intermittent catheter.

In some embodiments, the method comprises the step of adding at least one cross-linking activator to one or both of the base polymer and the additive.

The method may comprise adding both at least one cross-linking activator and at least one cross-linker to one or both of the base polymer and the additive.

The at least one cross-linking activator may activate cross-linking of the base polymer, the additive, and/or of the base polymer with the additive.

The at least one cross-linking activator may comprise a radical initiator. The radical initiator may comprise a thermal radical initiator and/or a photo-radical initiator. The radical initiator may comprise a peroxide. The peroxide may be selected from the group comprising: benzoyl peroxide (BPO), di-tent-butyl peroxide, cumene hydroperoxide, tert-butyl hydroperoxide, 2,5-bis(tert-butylperoxy)-2,5-dimethylhexane (DHBP), di(tert-butylperoxyisopropyl)benzene, dicumyl peroxide (DCP), 2,5-di(tert-butylperoxy)-2,5-dimethyl-3-hexyne (DTBPHY) or combinations and/or derivatives thereof. The radical initiator may comprise an azo compound. The azo compound may be selected from the group comprising: AIBN, AMBN, ADVN, ACVA, dimethyl 2,2′-azobis(2-methylpropionate), AAPH, and 2,2′-azobis[2-(2-imidazolin-2-yl)-propane] dihydrochloride, or combinations and/or derivatives thereof The photo-radical initiator may be selected from the group comprising: camphorquinone, acetophenone, 3-acetophenol, 4-acetophenol, benzophenone, 2-methylbenzophenone, 3-methylbenzophenone, 3-hydroxybenzophenone, 3,4-dimethylbenzophenone, 4-hydroxybenzophenone, 4-benzoylbenzoic acid, 2-benzoylbenzoic acid, methyl 2-benzoylbenzoate, 4,4′-dihydroxybenzophenone, 4-(dimethylamino)-benzophenone, 4,4′-bis(dimethylamino)-benzophenone, 4,4′-bis (diethylamino)-benzophenone, 4,4′-dichlorobenzophenone, 4-(p-tolylthio)benzophenone, 4-phenylbenzophenone, 1,4-dibenzoylbenzene, benzil, 4,4′-dimethylbenzil, p-anisil, 2-benzoyl-2-propanol, 2-hydroxy-4′-(2-hydroxyethoxy)-2-methylpropi ophenone (Irgacure 2959), 1-benzoylcyclohexanol, benzoin, anisoin, benzoin methyl ether, benzoin ethyl ether, benzoin isopropyl ether, benzoin isobutyl ether, o-tosylbenzoin, 2,2-diethoxyacetophenone, benzil dimethylketal, 2-methyl-4′-(methylthio) morpholinopropiophenone, 2-benzyl-2-(dimethylamino)-4′-morpholinobutyrophenone, 2-isonitrosopropiophenone, anthraquinone, 2-ethylanthraquinone, sodium anthraquinone-2-sulfonate, 9,10-phenanthrenequinone, 9,10-phenanthrenequinone, dibenzosuberenone, 2-chlorothioxanthone, 2-isopropylthioxanthone, 2,4-diethylthioxanthen-9-one, 2,2′-bis (2-chlorophenyl)-4,4′,5,5′-tetraphenyl -1,2′ -biimidazole, diphenyl(2,4,6-trimethylbenzoyl)phosphine oxide, phenylbis(2,4,6-trimethylbenzoyl)phosphine oxide, and lithium phenyl(2,4,6-trimethylbenzoyl)phosphinate, or combinations and/or derivatives thereof.

The free radical initiator may be present at 0.01 to 5 w %, 0.5 to 2 wt % based on the total weight of the monomers used to prepare the crosslinked polymer disclosed herein,

The molar ratio of free radical initiator to the reagent living/controlled polymerisation radical agent may range from 0.05 to 1:1, or 0.2:1 to 0.8:1 or 0.3 to 0.5:1.

The method may be carried out as a batch process, a semi-batch process, a continuous process, a feed process or a bulk process. The process may be in an emulsion, in solution or suspension.

The method may comprise adding the at least one cross-linking activator to one or both of the base polymer and the additive before or during extrusion or injection moulding of the intermittent catheter.

The method may comprise adding the at least one cross-linker to one or both of the base polymer and the additive before extrusion or injection moulding of the intermittent catheter and adding the at least one cross-linking activator to one or both of the base polymer and the additive after extrusion or injection moulding. This may comprise soaking the extruded or injection moulded intermittent catheter in a peroxide-containing solution, allowing the peroxide to diffuse into the catheter.

The method may comprise packaging the intermittent catheter in a container comprising a solution comprising the at least one cross-linking activator to induce cross-linking.

In some embodiments, the method comprises adding both MA cross-linker and DHBP cross-linking activator to one or both of the base polymer and the additive.

In some embodiments, the method comprises adding both HO-TEMPO cross-linker and di(tert-butylperoxyisopropyl)benzene cross-linking activator to one or both of the base polymer and the additive.

In some embodiments, the method comprises adding both BzO-TEMPO cross-linker and di(tert-butylperoxyisopropyl)benzene cross-linking activator to one or both of the base polymer and the additive.

In some embodiments, the method comprises adding both TMPTA cross-linker and DHBP cross-linking activator to one or both of the base polymer and the additive.

In some embodiments, the method comprises adding at least one cross-linking co-monomer to one or both of the base polymer and the additive.

The method may comprise adding both at least one cross-linking co-monomer and at least one cross-linker; both at least one cross-linking co-monomer and at least one cross-linking activator; or the method may comprise adding at least one cross-linking co-monomer, at least one cross-linker, and at least one cross-linking activator to one or both of the base polymer and the additive.

The at least one cross-linking co-monomer may be selected from the group comprising: an N-halosuccinimide (such as N-bromosuccinimide), a furan derivative, and butyl 3-(2-thienyl) propenoate (BTA), or combinations and/or derivatives thereof. The furan derivative may comprise a furan nitrile derivative, which may comprise 2-(furan-2-ylmethylene)malononitrile (FN).

The method may comprise adding the at least one cross-linking co-monomer to one or both of the base polymer and the additive before extrusion or injection moulding of the intermittent catheter. Adding at least one cross-linking co-monomer to one or both of the base polymer and the additive may assist in limiting chain scission of the base polymer and/or the additive leading to improved catheter material properties.

In some embodiments, the method comprises the step of irradiating one or both of the base polymer and the additive.

The method may comprise irradiating with E-beam and/or γ radiation.

The method may comprise irradiating one or both of the base polymer and the additive after addition of one or more of the groups comprising: the at least one cross-linker, the at least one cross-linking activator, the at least one cross-linking co-monomer, and combinations thereof.

The method may comprise irradiating the intermittent catheter after extrusion or injection moulding to cross-link the catheter surface. Irradiating one or both of the base polymer and the additive may sterilise the intermittent catheter as well as encourage cross-linking.

Irradiating for sterilisation and cross-linking at the same time may be advantageous in reducing the number of operations required in manufacturing the intermittent catheter.

The method may comprise adding at least one inorganic additive or filler to one or both of the base polymer and the additive before irradiation cross-linking. The inorganic additive or filler may be selected from the group comprising: TiO₂, CaCO₃, talc, clay, and combinations thereof. Inorganic additives and fillers are believed to provide the advantage of increasing the number of radicals generated during irradiation cross-linking and/or after UV exposure.

In some embodiments, the method comprises the steps of grafting silane groups to the base polymer; and adding water to the base polymer to cross-link the silane groups.

The base polymer may preferably comprise a polyolefin.

The step of grafting silane groups to the base polymer may comprise reacting the base polymer with an organofunctional silane.

The organofunctional silane may comprise one or more functional groups of the list comprising: an unsaturated group, a thiol, an amine, and an epoxide. The organofunctional silane may be selected from the group comprising: vinyltrimethoxysilane (VMSI), vinyltriethoxysilane (VESI), 3-methacryloyloxypropyl-trimethoxysilane (MMSI), 3-mercaptopropyl-trimethoxysilane (SMSI), 3-aminopropyl-trimethoxysilane (NMSI), and 3-glycidyloxypropyl-trimethoxysilane (GMSI), or combinations and/or derivatives thereof.

The method may comprise adding the organofunctional silane in a concentration of at least 0.1, 0.2, 0.3, 0.4, 0.5, 0.75, 1, 2, 3, 4, 5, 6, 7, 8, 9, or at least 10% by weight of the base polymer. The method may comprise adding the organofunctional silane in a concentration of no more than 40, 35, 30, 25, 20, or 15% by weight of the base polymer. A preferred concentration may be between 0.1% and 10% by weight of the base polymer, and more preferably between 1% and 5%, or at a concentration of 2% by weight of the base polymer.

The reaction of the base polymer with the organofunctional silane may be performed in the presence of the at least one cross-linking activator. The at least one cross-linking activator may comprise a radical initiator as listed above.

The method may comprise adding the at least one cross-linking activator in a concentration of at least 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or at least 2% by weight of the base polymer. The method may comprise adding the at least one cross-linking activator in a concentration of no more than 10% by weight of the base polymer, or no more than 9, 8, 7, 6, 5, 4, or 3% by weight of the base polymer. A preferred concentration may be between 0.01% and 2% by weight of the base polymer, and more preferably between 0.05% and 1%, or at a concentration of around 0.1% to 0.5% by weight of the base polymer.

The step of grafting silane groups to the base polymer may be performed during extrusion or injection moulding of the base polymer.

The step of grafting silane groups to the base polymer may be performed at an elevated temperature of at least 50° C., or at least 60, 70, 80, 90, 100, 110, 120, 130, 140, 150, 160, 170, 180, 190, 200, 210, 220, 230, 240, 250, 260, 270, 280, 290, or at least 300° C. This step may be performed at a temperature of between 100° C. and 300° C., and preferably between 140° C. and 240° C.

The step of cross-linking the silane groups may be performed by adding water after extrusion or injection moulding of the base polymer.

The step of adding water to the base polymer to cross-link the silane groups may be performed at a temperature of at least 20° C., or at least 30, 40, 50, 60, 70, 80, 90, 100, 110, 120, 130, 140, or at least 150° C. This step may be performed at a temperature of between 50° C. and 150° C., preferably between 60° C. and 90° C.

The step of adding water to the base polymer may be performed in a steam chamber of a hot water tank.

The step of cross-linking the silane groups may comprise adding a catalyst, preferably before adding water to the base polymer. The catalyst may be added during extrusion or injection moulding of the base polymer. The catalyst may comprise an organotin derivative, such as dibutyltindilaurate.

The silane groups may be cross-linked for at least 5, 10, 15, 20, 25, or at least 30 minutes, or for at least 1, 2, 3, 4, 5, 6, 7, 8, 9, 10, 12, 14, 16, 18, 20, or at least 22 hours, or for at least 1 day, or at least 1 day and 5, 10, 15, 20 hours, or for at least 2 days.

In some embodiments, the base polymer is functionalised with reactive side chains and the method comprises the step of cross-linking the base polymer independently and/or with the additive by reacting the reactive side chains with each other and/or with functional groups on the additive.

Cross-linking may be performed by irradiating the functionalised base polymer, preferably with ultraviolet radiation.

Cross-linking may be performed at a temperature of at least 10° C., or at least 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or at least 400° C. Cross-linking may be performed at a temperature of between 20° C. and 300° C., preferably between 30° C. and 200° C.

Cross-linking may be performed for at least 5, 10, 15, 20, 25, or at least 30 minutes, or for at least 1, 2, 3, 4, 5, 6, 7, 8, 9, 10, 12, 14, 16, 18, 20, or at least 22 hours, or for at least 1 day, or at least 1 day and 5, 10, 15, 20 hours, or for at least 2 days.

The functionalised base polymer may preferably comprise a polyolefin functionalised with reactive side chains. The reactive side chains may be selected from the group comprising: an unsaturated group, an epoxide, a borane, and combinations thereof. The functionalised base polymer may comprise a polyolefin co- or terpolymer comprising the reactive side chains. The reactive side chains may be incorporated onto the polyolefin chain through a metallocene catalysed reaction using reactive co-monomers comprising the reactive side chains. The reactive co-monomer may be selected from the group comprising: a vinylbenzene derived olefin, a 9-BBN derived olefin, a methylbenzene derived olefin, a glycidyl derived olefin, and combinations thereof. The functionalised base polymer may be selected from the group comprising: poly(ethylene-ter-propylene-ter-divinylbenzene) (EP-DVB), poly(ethylene-ter-1-octene-ter-divinylbenzene) (EO-DVB), poly(ethylene-co-glycidyl methacrylate), and combinations thereof.

In some embodiments, the additive comprises poly(alkylene oxide) groups and the method comprises the step of forming non-covalent bonds between the poly(alkylene oxide) groups and a complexing agent to cross-link the additive.

The complexing agent may be selected from the group comprising: a urea, a cyclodextrin, and a poly(unsaturated carboxylic acid), or combinations and/or derivatives thereof. The urea is preferably urea per se. The cyclodextrin may be selected from the group comprising: α-cyclodextrin, β-cyclodextrin, and γ-cyclodextrin; preferably α-cyclodextrin. The poly(unsaturated carboxylic acid) may preferably comprise poly(methacrylic acid) or a copolymer thereof. The copolymer may comprise a copolymer of poly(methacrylic acid) and an acrylate polymer, and preferably poly(methacrylic acid-co-methyl methacrylate).

The method may comprise packaging the intermittent catheter in a container comprising a solution of the complexing agent to form the non-covalent bonds between the poly(alkylene oxide) groups and the complexing agent.

According to a fourth aspect of the invention there is provided a packaged intermittent catheter of the first aspect of the invention comprising a packaging container in which is located an intermittent catheter of the first aspect of the invention, and optionally a wetting agent.

The wetting agent may surround the intermittent catheter or may be separated from the intermittent catheter within the packaging, for example by providing the wetting agent in a separate container within the packaging container.

The wetting agent may comprise an aqueous solution or water. The aqueous solution may comprise one or more ingredients selected from the group comprising a salt, a buffer, an antibiotic, an active agent (which may be a medicament) and a thickening agent.

### DETAILED DESCRIPTION OF THE INVENTION

In order that the invention may be more clearly understood embodiments thereof will now be described, by way of example only:

### Example 1

A first embodiment of an intermittent catheter of the invention comprises an intermittent catheter comprising a hollow polymeric tubular body comprising a base polymer formed of thermoplastic polypropylene and further comprising an amphiphilic additive of the formula CH₃CH₂(CH₂CH₂)₁₅(OCH₂CH₂)₅OH. The amphiphilic additive comprises a hydrophilic block which seeks towards the outer surface of the body due to its incompatibility with the base polymer, the outer surface becoming lubricious as a result. The lipophilic and hydrophobic block of the amphiphilic additive ensures that the hydrophilic block is secured to the base material. The amphiphilic additive is independently cross-linked through non-covalent bonds formed between the poly(ethylene oxide) groups and a urea complexing agent.

The non-cross-linked intermittent catheter may be prepared as described in US patents U.S. Pat. Nos. 10,058,638 B2 and 9,186,438 B2. The amphiphilic additive may be independently cross-linked by treating the non-cross-linked intermittent catheter with a solution of urea.

The intermittent catheter is used in the conventional manner.

The amphiphilic additive at the outer surface of the intermittent catheter body confers high lubricity to the outer surface of the intermittent catheter, making it both easier to insert and remove. The additive cross-links increase the difficulty for the additive to migrate, particularly out of the intermittent catheter. Cross-linking the additive is believed to reduce the mobility of the additive within the polymer matrix through creating an insoluble matrix of additive and/or base polymer, which in turn restricts the migration of the additive out of the catheter. This allows for the intermittent catheter to retain its lubricious surface for longer and even when the surface of the catheter is scraped or when the catheter is packaged in water or aqueous solutions.

The intermittent catheter of Example 1 conferred reduced migration of the amphiphilic additive from the surface of the catheter during both storage/transport and through use of the catheter. It also provided reduced resistance to abrasion of the additive from the surface of the catheter on contact with external bodies.

### Example 2

A second embodiment of an intermittent catheter of the invention comprises an intermittent catheter comprising a hollow polymeric tubular body comprising a base polymer formed of polyethylene and further comprising an amphiphilic additive of the formula CH₃CH₂(CH₂CH₂)₂₀(OCH₂CH₂)₈OH on the outer surface of the body. The amphiphilic additive is cross-linked with the base polymer.

The non-cross-linked intermittent catheter may be prepared as described in US patents

U.S. Pat. Nos. 10,058,638 B2 and 9,186,438 B2, but without the step of adding the additive. The non-cross-linked intermittent catheter may then be treated with a mixture of the amphiphilic additive, PEGDA multifunctional monomer and Irgacure 2959 photoinitiator followed by ultraviolet irradiation. This provides cross-linking of the amphiphilic additive to the base polymer on the outer surface of the intermittent catheter.

The intermittent catheter is used in the conventional manner.

The amphiphilic additive on the outer surface of the intermittent catheter body confers high lubricity to the outer surface of the intermittent catheter, making it both easier to insert and remove. Cross-linking the additive to the base polymer increases the difficulty for the additive to migrate, particularly out of the intermittent catheter. This allows for the intermittent catheter to retain its lubricious surface for longer and even when the surface of the catheter is scraped or when the catheter is packaged in water or aqueous solutions. The intermittent catheter of Example 1 conferred reduced migration of the amphiphilic additive from the surface of the catheter during both storage/transport and through use of the catheter. It also provided reduced resistance to abrasion of the additive from the surface of the catheter on contact with external bodies.

### Example 3

A third embodiment of an intermittent catheter of the invention comprises an intermittent catheter comprising a hollow polymeric tubular body comprising a base polymer formed of thermoplastic polyethylene and further comprising an amphiphilic additive of the formula CH₃CH₂(CH₂CH₂)₁₀(OCH₂CH₂)₄OH. The amphiphilic additive comprises a hydrophilic block which seeks towards the outer surface of the body due to its incompatibility with the base polymer, the outer surface becoming lubricious as a result.

The lipophilic and hydrophobic block of the amphiphilic additive ensures that the hydrophilic block is secured to the base material. The thermoplastic polyethylene base polymer comprises silane groups and the base polymer is independently cross-linked through Si—O—Si bonds between the silane groups and water.

The non-cross-linked intermittent catheter may be prepared as described in US patents U.S. Pat. Nos. 10,058,638 B2 and 9,186,438 B2, but with an added step of adding VMSI organofunctional silane and DCP peroxide to the polymer mixture during extrusion of the intermittent catheter to graft the silane to the polyethylene base material. The non-cross-linked intermittent catheter may then be treated with water in a steam chamber after extrusion. This effects independent cross-linking of the base polymer.

The intermittent catheter is used in the conventional manner.

The amphiphilic additive at the outer surface of the intermittent catheter body confers high lubricity to the outer surface of the intermittent catheter, making it both easier to insert and remove. The base polymer cross-links increase the difficulty for the additive to migrate, particularly out of the intermittent catheter. This allows for the intermittent catheter to retain its lubricious surface for longer and even when the surface of the catheter is scraped or when the catheter is packaged in water or aqueous solutions. The intermittent catheter of Example 1 conferred reduced migration of the amphiphilic additive from the surface of the catheter during both storage/transport and through use of the catheter. It also provided reduced resistance to abrasion of the additive from the surface of the catheter on contact with external bodies.

The above embodiments are described by way of example only. Many variations are possible without departing from the scope of the invention as defined in the appended claims.

1. An intermittent catheter comprising a hollow polymeric tubular body comprising a base polymer and further comprising an amphiphilic additive at and/or on an outer surface of the body, wherein one or both of the base polymer and the additive are independently cross-linked and/or the base polymer and additive are cross-linked with each other.

2. An intermittent catheter as claimed in claim 1, wherein the base polymer is cross-linked.

3. An intermittent catheter as claimed in claim 1, wherein the additive is cross-linked.

4. An intermittent catheter as claimed in claim 1, wherein the base polymer and the additive are cross-linked with each other.

5. An intermittent catheter as claimed in claim 1, wherein the base polymer is cross-linked at and/or on the outer surface.

6. (canceled)

7. (canceled)

8. An intermittent catheter as claimed in claim 1, wherein the additive is an amphiphilic A-B block copolymer comprising an A-block comprising a hydrophobic hydrocarbon portion and a hydrophilic B-block.

9. An intermittent catheter as claimed in claim 8, wherein the additive is an A-B block copolymer comprising an A-block comprising a hydrocarbon chain block of the formula CH₃CH₂(CH₂CH₂)ₐ where “a” is 5-25, and a hydrophilic B-block.

10. (canceled)

11. An intermittent catheter as claimed in claim 1, wherein the additive comprises poly(alkylene oxide) groups and the additive is cross-linked through non-covalent bonds between the poly(alkylene oxide) groups and a complexing agent.

12. An intermittent catheter as claimed in claim 1, wherein the additive is at and/or on at least 50% of the outer surface area of the polymeric tubular body.

13. A method of manufacturing an intermittent catheter, the method comprising the steps of:
a. providing a base polymer and an amphiphilic additive; and
b. forming a hollow polymeric tubular catheter body comprising the base polymer and additive
wherein the method further comprises the step of independently cross-linking one or both of the base polymer and additive and/or cross-linking the base polymer and the additive with each other.

14. A method as claimed in claim 13, wherein the method comprises cross-linking the base polymer.

15. A method as claimed in claim 13, wherein the method comprises cross-linking the additive.

16. A method as claimed in claim 13, wherein the method comprises cross-linking the base polymer and the additive with each other.

17. A method as claimed in claim 13, wherein the method comprises cross-linking one or both of the base polymer and the additive independently, before cross-linking the base polymer and the additive with each other.

18. A method as claimed in claim 13, wherein the method comprises cross-linking one or both of the base polymer and the additive independently, after cross-linking the base polymer and the additive with each other.

19. A method as claimed in claim 13, wherein the method comprises cross-linking one or both of the base polymer and the additive independently and/or cross-linking the base polymer and the additive with each other during co-granulation of the base polymer with the additive.

20. A method as claimed in claim 13, wherein the method comprises the step of cross-linking one or both of the base polymer and the additive independently and/or cross-linking the base polymer and the additive with each other during and/or after extrusion or injection moulding of a mixture comprising the base polymer and the additive.

21. A method as claimed in claim 13, wherein the method comprises the step of cross-linking one or both of the base polymer and the additive independently and/or cross-linking the base polymer and the additive with each other during and/or after co-extrusion of a layer or coating of the additive on an outer surface of the base polymer.

22. (canceled)

23. (canceled)

24. A method as claimed in claim 13, wherein the additive is an amphiphilic A-B block copolymer comprising an A-block comprising a hydrophobic hydrocarbon portion and a hydrophilic B-block.

25. A method as claimed in claim 24, wherein the additive is an A-B block copolymer comprising an A-block comprising a hydrocarbon chain block of the formula CH₃CH₂(CH₂CH₂)ₐ where “a” is 5-25, and a hydrophilic B-block.

26-31. (canceled)
